(19) United States
(12) Patent Application Publication
(10) Pub. No.: US 2005/0009199 A1
(43) Pub. Date: Jan. 13, 2005

(54) Method of measuring number of molecules or molecular density of a sample fixed on a substrate surface

(75) Inventor: Taigo Nishida, Kyoto-fu (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: Shimadzu Corporation, Kyoto (JP)

(21) Appl. No.: 10/875,175

(22) Filed: Jun. 25, 2004

(30) Foreign Application Priority Data
Jul. 7, 2003 (JP) 2003-271637(P)

(51) Int. Cl. C12Q 1/68

(52) U.S. Cl. 436/172

(57) ABSTRACT

The method of measuring the number of molecules or the molecular density of a sample according to the present invention includes the steps of: a) labeling each of the molecules with a preset number of fluorescent substances before or after the sample is fixed on the substrate surface, b) obtaining an image of the fluorescent substances from the whole of the substrate surface or from a part of the substrate surface, c) measuring the number of the fluorescent spots and the intensity of every fluorescent spot, and d) calculate the number of molecules or the density (concentration) of molecules of the sample based on the above-described measurement results.

The present invention relates to a method of fixing a sample of protein, DNA or the like on a substrate surface, and of quantitatively measuring the number of molecules, and the density or the concentration of the molecules of the sample.

### BACKGROUND OF THE INVENTION

A variety of biological samples such as proteins, DNAs, peptides, saccharides, etc. are analyzed and measured in the fields of biochemistry, molecular biology, clinical medicine or other various fields. In one of such measurements, a biological sample of protein, DNA, etc. is fixed on a plate substrate, an excitation light is irradiated onto the sample, and the fluorescent light emitted from the sample is detected. Paragraph 0004-0006 of the Unexamined Japanese Patent Application Publication No. 2002-214232 describes the method.

In order to measure the molecular density of a fixed sample using fluorescent light, the following method is known. Fluorescent intensities of several standard samples with known densities (or concentrations) are measured using a fluorescent imager, a fluorescent spectrophotometer or the like, and a calibration curve is made to represent the relationship between the density and the intensity of the fluorescent light. Then an unknown sample is tested under the same condition as the standard samples, and the intensity of the fluorescent light is applied to the calibration curve, whereby the molecular density of the sample is measured.

In the above method, standard samples of known densities are necessary, but it is very troublesome to prepare many standard samples for various testing conditions. Especially, it is difficult to prepare standard samples that are stable and have very small dilution errors. The test conditions at the time of testing standard samples and at the time of testing unknown samples must be the same to obtain reliable measurement results, but it is actually difficult to achieve.

### SUMMARY OF THE INVENTION

An object of the present invention is therefore to provide a method of measuring the molecular density (or molecular concentration) of a sample fixed on a substrate surface without using standard samples of known densities.

According to the present invention, the method includes the steps of:
a) labeling each of the molecules with a preset number of fluorescent substances before or after the sample is fixed on the substrate surface,
b) obtaining an image of the fluorescent substances from the whole of the substrate surface or from a part of the substrate surface,
c) measuring the number of the fluorescent spots and the intensity of every fluorescent spot, and
d) calculate the number of molecules or the density (concentration) of molecules of the sample based on the above-described measurement results.

In the method of the present invention, the monomolecular fluorometric imaging method is introduced. The monomolecular fluorometric imaging method has been used primarily for the purpose of observing and tracing a certain object molecule in a living body. In the present method, the monomolecular fluorometric imaging method is used to count the number of molecules. In labeling the molecules in the present method, a preset number of fluorescent substances are affixed to each of the molecules, wherein the preset number includes one. In many cases, one or two fluorescent substances per molecule is adequate for the present method. The fluorescent substance (or substances) can be affixed directly to a molecule. Otherwise, the preset number of fluorescent substance or substances are first affixed to another substance such as an appropriate antibody that reacts specifically with the object sample, and then the another substance is reacted with the object sample (an indirect labeling method).

The above-described labeling process can be done after the sample is fixed to the substrate surface. But it is generally more convenient if it is done before the sample is fixed to the substrate surface. The fixing method is not limited to a specific one. An example is to promote fixing by modifying the substrate surface with reactive functional group that can bind with the sample, and another example is fixing with a nonspecific adsorption.

Using the monomolecular fluorometric imaging method, it is basically possible to obtain the number of molecules by counting the fluorescent spots. However, it sometimes happen that plural fluorescent spots are counted as one spot because they are situated very close to one another even when the molecular concentration is rather low and the distribution of the fluorescent spots is thin. When plural fluorescent spots overlap to form a spot, the fluorescent intensity of the overlapped spot is usually stronger than that in the case of a simple spot. Thus it is possible to correct the molecular counting and enhance the exactness of the count (or correctness of the number of molecules) by considering the fluorescent intensity of the spots.

It is possible to further enhance the correctness of the counting of molecules by introducing a process or operation to deliberately promote the decrease of fluorescent light of the fluorescent substance. For example, first the initial number of fluorescent spots and the initial intensity of every spot are measured on the image. Then the decrease of the fluorescent light of the fluorescent substance is promoted. The number of molecules or the molecular density is calculated based on the initial number and initial intensity taking account of the changing manner of the intensity of the fluorescent light, the changing manner of the number of fluorescent spots, or both in the course of the decrease or after the decrease of the fluorescent light. In the process of the decrease of the fluorescent light, the fluorescent intensity weakens stepwisely according to the number of molecules constituting an initial fluorescent spot. The information of such a changing manner of fluorescent intensity can be used to correct the number of molecules.

When the distribution of the fixed sample is dense, fluorescent light spots of distinct molecules that are situated close to one another overlap everywhere so that they are not looked as spots but it seems that a broad area or even the whole substrate surface glows. In such a case, the initial counting of spots is difficult. But the method of promoting decrease of the fluorescent light is effective in this case to decrease the density of the fluorescent light spots on the substrate surface.

For example, an initial fluorescent light intensity is measured based on the image, and the decrease of the fluorescent light of the fluorescent substance is promoted. After the decrease, the number of fluorescent lights spots and the fluorescent light intensity are measured. The number of molecules or the molecular density is calculated from the initial fluorescent light intensity referring to the relationship between the number of fluorescent light spots and their intensity after the decrease. When the fluorescence density decreases, one or several spots among those indivisible before the decrease vanishes and individual fluorescent spots become distinct. When the decrease is further promoted, a fluorescent spot of a molecule is clarified. This enhances the strict relationship between the number of fluorescent spots and the number of molecules. By measuring the fluorescent intensity and the number of fluorescent spots in such a state, the initial number of fluorescent spots corresponding to the actual number of molecules can be estimated from the initial fluorescent intensity, which enables obtaining the number of molecules.

It is preferable to measure the fluorescent intensity and the number of fluorescent spots at least twice when the decrease of the fluorescent light is promoted, and a calibration curve (or line) is drawn with the results. Referring to the calibration curve, the initial number of fluorescent spots can be determined (or estimated) from the initial fluorescent intensity. This enhances the correctness of the estimation of the number of fluorescent spots.

An effective method of decreasing the fluorescent light of fluorescent substances is to irradiate a laser beam onto the fixed sample, which results in quenching of the fluorescent light. Another method is to promote suppression of the fluorescent light using the chemical quenching method.

According to the method of the present invention, it is possible to measure the number of molecules or the molecular density of an object sample such as protein or DNA, for example, at high precision without measuring a standard sample of known density (or concentration). Thus a burdensome preparation of standard samples is not necessary, and the measurement is facilitated. It is not necessary either to adjust the measuring conditions of an unknown sample and a standard sample, which alleviates the burden of controlling the measuring conditions and improves the measuring efficiency.

### BRIEF DESCRIPTION OF THE DRAWINGS

FIG. 1 is a flowchart of a measuring method embodying the present invention.

FIG. 2 is a schematic diagram of a measuring system using a total reflection fluorescent microscope.

FIG. 3 is an example of a calibration curve.

FIG. 4 is an example of a fluorescence image (fluorescence Cy-5).

FIG. 5 is a graph showing the change in the intensity of a fluorescent spot with respect to time.

FIG. 6 is a histogram showing the distribution of intensities of fluorescent spots of an entire image.

FIG. 7 is a graph showing the correlation between the decrease in the number of fluorescent spots and the decrease in the average fluorescent intensity due to quenching.

### DETAILED DESCRIPTION OF A PREFERRED EMBODIMENT

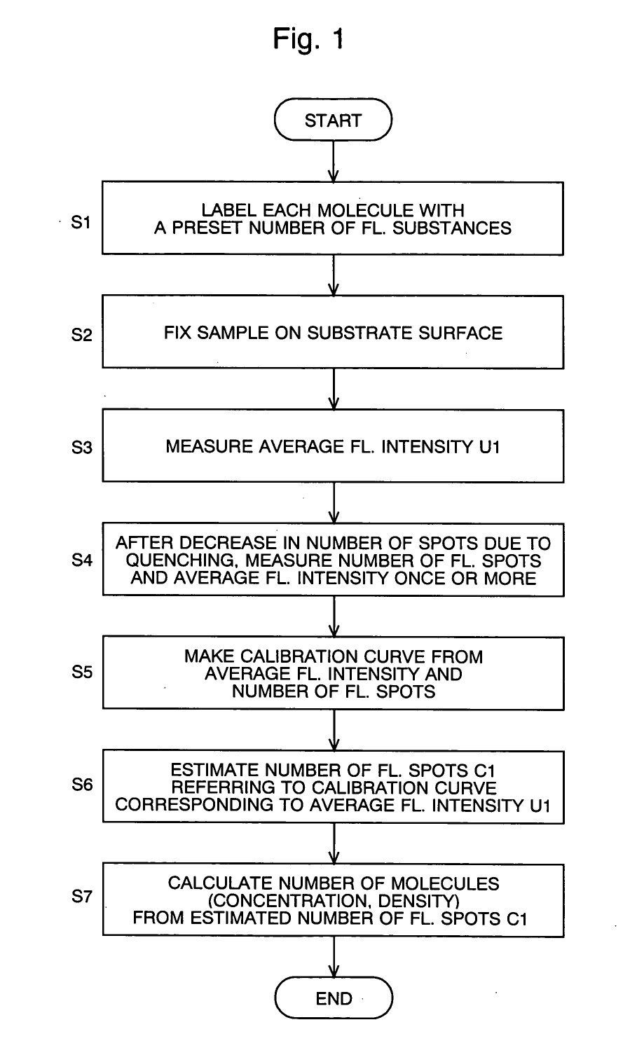

A method of measuring the molecular density embodying the present invention is described referring to the flowchart of FIG. 1. First, a sample, such as protein or DNA, for example, is labeled so that every molecule is labeled by a preset number of fluorescent substances (Step S1). Normally, the preset number is one. The labeling method and the fluorescent substance are chosen according to the kind of the sample. The labeled sample is fixed on a substrate surface (Step S2). Known fixing methods can be used here. Thus the sample is prepared.

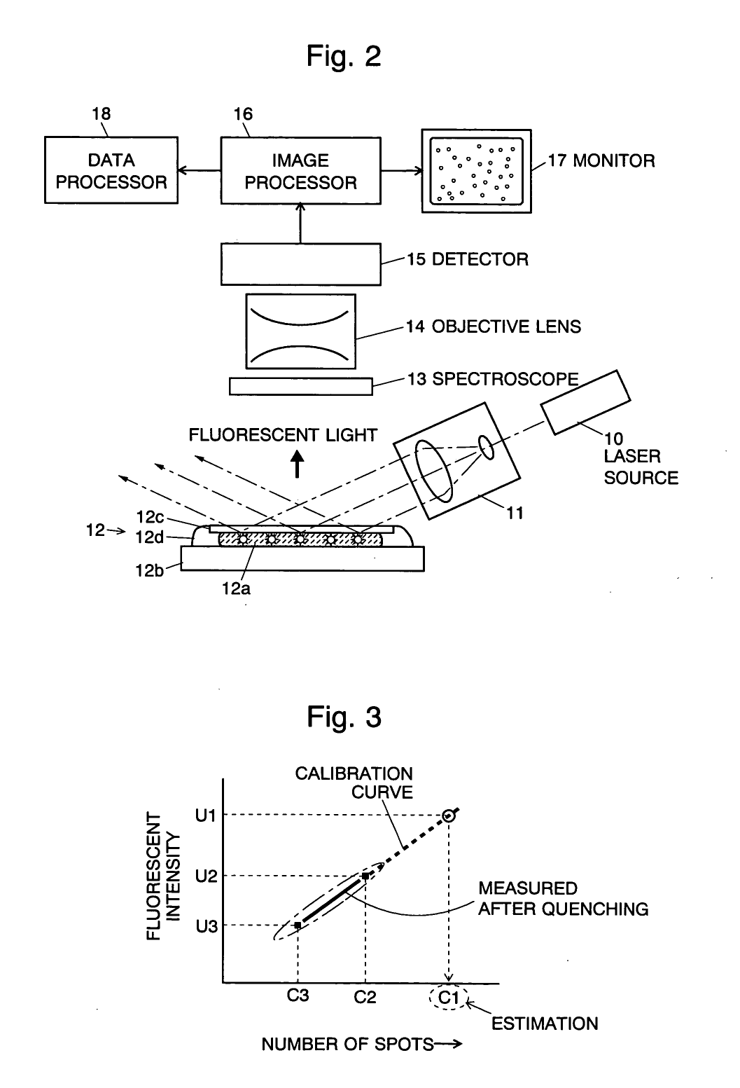

Then the sample is set on a fluorescent measuring device. Since fluorescent light is generally weak, it is necessary to obtain individual spots from the background light and measure the fluorescent light with high sensitivity. A total reflection fluorescent microscope or a confocal microscope is suitable for such a measurement. FIG. 2 is a schematic diagram of a measuring system using a total reflection fluorescent microscope. Its structure and operation is described using FIG. 2.

A sample unit 12 is composed of a sample 12a fixed on a substrate 12b made of quartz glass or the like, where the sample 12a is the above-described labeled sample. The sample 12a is sealed with a cover glass 12c and a sealant 12d such as manicure. A laser beam emitted from a laser source 10 is irradiated through a lens unit 11 on the sample unit 12. In the sample unit 12, the laser beam enters the measurement plane, which is the border between the cover glass 12c and the sample 12a, with the incident angle larger than the critical angle θ, where the critical angle θ is determined from the refraction indices of the cover glass 12c and the sample 12a. The incident laser beam is totally reflected at the measurement plane, but a small part of the light leaks into the sample 12a owing to the near-field effect, and the near-field light excites fluorescent substances in the sample 12a in the vicinity of the cover glass 12c. The fluorescent substances emit spontaneous fluorescent light.

The fluorescent light is detected by the detector 15 through the spectroscope 13 and the objective lens 14. The spectroscope 13 is endowed with a function to pass only light of a predetermined wavelength. In some cases, the spectroscope 13 can be omitted. The fluorescent light is weak, as mentioned before, it is preferable to use a detector 15 having high sensitivity. For example, a unit of a cooled CCD sensor and an image intensifier tube is recommended. The image signal generated in the detector 15 is sent to the image processor 16, where a two-dimensional image is composed and shown on the display monitor 17. The two-dimensional image signal is also sent to the data processor 18, where the data is processed to calculate the molecular density of the sample as described later.

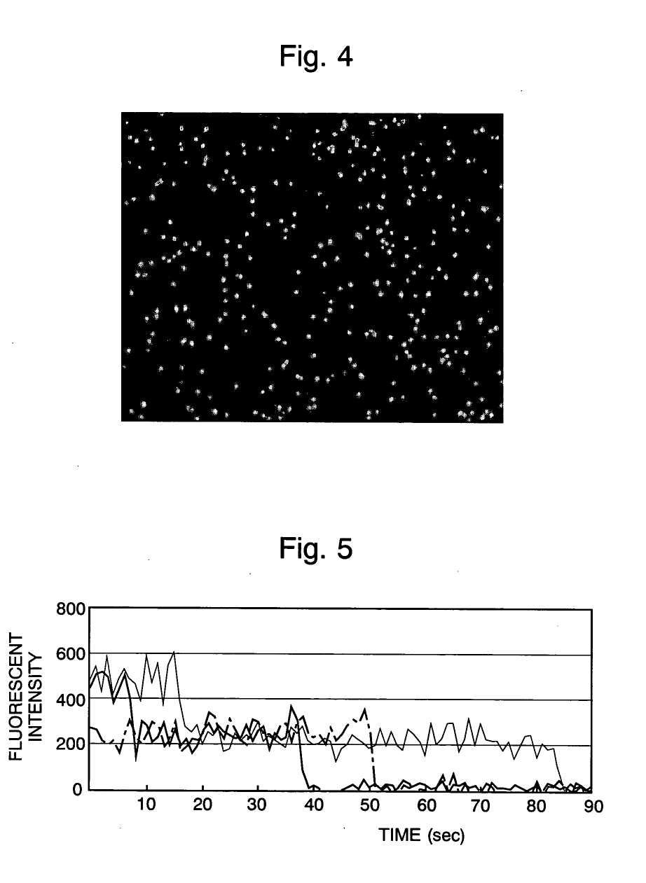

Returning to FIG. 1, the detector 15 generates a signal of an image including many fluorescent spots (as shown in FIG. 4) just after the laser beam irradiation of the sample unit 12 is started. The data processor 18 calculates the average fluorescent intensity U1 of the whole image (or a restricted area of the image) based on the image signal. As shown in FIG. 4, many fluorescent spots appear on the image. A fluorescent spot does not necessarily represent a molecule even when every molecule is labeled by a fluorescent substance, because very closely located or overlapped fluorescent substances affixed to respective molecules can be looked and detected as a fluorescent spot if the molecular density is rather high. The count of the fluorescent spots at this stage does not necessarily reflect the true number of molecules.

Thus it is awaited until the number of fluorescent spots decreases, and a second measurement is made. Since it takes a long time until the intensity of the fluorescent light naturally decreases, the intensity is forcefully decreased. For example, it is known that the above-described fluorescent substances stop emitting light (or are quenched) when they are irradiated laser light for a certain period. Since the time until a fluorescent substance are quenched shows a certain stochastic distribution, the number of fluorescent spots in an image decreases gradually as time passes and almost all the fluorescent spots disappear at a certain period later. In the quenching process, the overlapping or very closely located fluorescent spots gradually decrease, and the correspondence between a fluorescent spot and a fluorescent substance (or a molecule of the sample) becomes clearer.

Thus the detector 15 generates a signal of an image of fluorescent spots a predetermined period after the laser beam is irradiated on the sample unit 12, and the number (or density) of the fluorescent spots decreases due to quenching. Based on the image signal, the data processor 18 calculates the average intensity of the fluorescent light of the whole image (or of a restricted object area of the image), and counts the number of fluorescent spots in the image. It is preferable to perform the measurement at least twice (Step S4).

Suppose two measurements are made with a certain interval between them, and the average intensity of the fluorescent light are U2 and U3, and the number of fluorescent spots are C2 and C3. Based on the measurement results, the data processor 18 creates a calibration curve as shown in FIG. 3 (Step S5). If the numbers of fluorescent spots C2 and C3 represent the numbers of the fluorescent substances at the respective measurement times (i.e., if there is no influence of overlapping of the spots), and if the average fluorescent intensity and the number of the fluorescent spots have a linear relationship, it is possible to estimate the true number of fluorescent spots (i.e., the number after being corrected for the overlapping sites) using the calibration curve as shown in FIG. 3. Thus the data processor 18 consults with the calibration curve, and estimates the true number C1 of the fluorescent spots corresponding to the average fluorescent intensity U1 (Step S6).

Since the number of fluorescent substances affixed to a molecule was known when the fluorescent labeling was done, the number of molecules can be calculated from the number C1 of the fluorescent spots, and the molecular density or molecular concentration is calculated (Step S7). When a molecule was affixed by, or labeled with, a fluorescent substance, the number of fluorescent spots directly corresponds (or equal) to the number of molecules, whereby the number of molecules and the molecular density are most easily calculated. When two or more fluorescent substances are affixed to a molecule, the quenching occurs stepwisely several times, so that it is preferable to make a proper correction in calculating the number of molecules from the number of fluorescent spots using a quenching profile which show the quenching manner in relation to time.

When the proportional relationship between the average fluorescent intensity and the number of fluorescent spots is maintained after quenching, the number of spots before quenching can be calculated after quenching. In this case, it is sufficient to measure the relationship between the average fluorescent intensity and the number of fluorescent spots once after quenching. When the relationship between the average fluorescent intensity and the number of fluorescent spots is measured three times or more, a calibration curve of higher order function, rather than the linear one shown in FIG. 3, can be made. For the purpose of reducing the number of fluorescent spots, it is possible to use the chemical quenching method instead of the laser irradiation quenching.

### EXAMPLE 1

An example of measuring the density of DNA using the method of the present invention is described. The 5′-terminal of a DNA (20mer) is labeled with Cy-5, and the. 3′-terminal is labeled with Biotin. The substrate 12b is a quartz slide glass which has little background light. After cleansing the quartz slide glass, Biotin-BSA is non-specifically adsorbed, and Streptavidin is reacted. Using the fixing agent, the labeled DNA with a concentration of 100 pM is fixed on the substrate 12b. The sample 12a is air-tightly sealed with a cover glass 12c and a sealant 12d of manicure. A prism total-reflection fluorescent microscope is used to measure the fluorescence. A laser of 635 nm wavelength is used as the exciting light, and its incident angle is set at 69°. A cooled CCD sensor with an image intensifier tube is used for the detector 15. The CCD sensor has the resolution of 200×200 pixels.

An example of the fluorescence image (Cy-5 fluorescence) obtained in the above measurement is shown in FIG. 4. The quenching manner in relation to time, or the quenching profile, of a fluorescent spot due to label Cy-5 when a laser beam is irradiated to the sample is shown in FIG. 5. Two different quenching manners are shown in this figure: one that the fluorescent intensity decreases to almost zero in a step, and the other that the fluorescent intensity decreases to almost zero in two steps. The former corresponds to the fluorescent spot of an initially single fluorescent substance or an initially single molecule, and the latter corresponds to the fluorescent spot of an initially two fluorescent substances or an initially two molecules. Thus the quenching profile reveals whether a fluorescent spot corresponds to a molecule or more molecules.

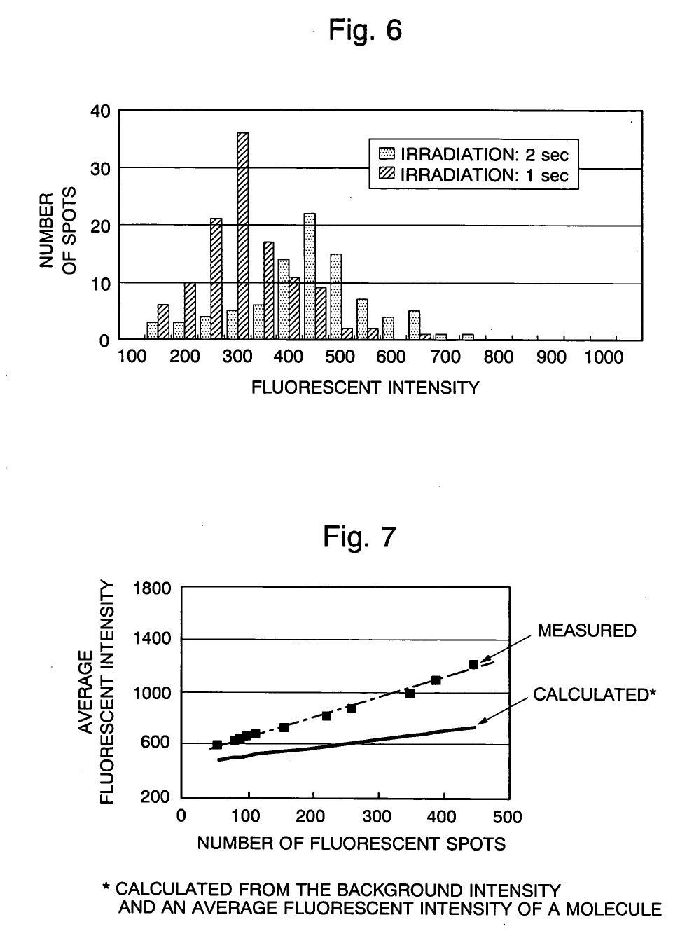

FIG. 6 is the histogram of the distribution of intensities of the fluorescent spots within a certain area of the fluorescent image. As seen from the figure, the distribution is almost the normal distribution. This means that the average fluorescent intensity of the area is influenced little by the variation of intensities of individual fluorescent spots, and that it is possible to distinguish between the spots of a molecule and two or more (practically two) molecules. In summary, from FIGS. 5 and 6, it is possible to distinguish the fluorescent spots of a molecule from two or more molecules using the quenching profile, and to calculate the number of fluorescent spots corresponding to the number of molecules from the average fluorescent intensity.

FIG. 7 is a graph of the relationship between the decrease in the fluorescent spots due to quenching and the decrease in the average fluorescent intensity, which shows a fairly good linearity between the average fluorescent intensity and the number of fluorescent spots after quenching. It is generally known that the number of fluorescence labeled molecules and the overall fluorescent intensity has a linear relationship, and it can be estimated at high probability that the linearity is maintained when the molecular density is high. Thus by using the calibration curve of the fluorescent spots after quenching and the average fluorescent intensity, the molecular density can be calculated rather exactly even when the molecular density is rather high.

Although only some exemplary embodiments of this invention have been described in detail above, those skilled in the art will readily appreciated that many modifications are possible in the exemplary embodiments without materially departing from the novel teachings and advantages of this invention. Accordingly, all such modifications are intended to be included within the scope of this invention.

1. A method of measuring a number of molecules or a molecular density of a sample fixed on a substrate surface comprising steps of:
labeling each molecule of the sample with a preset number of fluorescent substance or substances before or after the sample is fixed on the substrate surface;
obtaining an image of the whole substrate surface or a part of the substrate surface bearing fluorescent spots due to the fluorescent substances;
measuring a number of fluorescent spots and a fluorescent intensity of each of the fluorescent spots of the image; and
estimating a number of molecules or a molecular density of the sample based on the number of fluorescent spots and the fluorescent intensities of the fluorescent spots.

2. A method of measuring a number of molecules or a molecular density of a sample fixed on a substrate surface comprising steps of:
labeling each molecule of the sample with a preset number of fluorescent substance or substances before or after the sample is fixed on the substrate surface;
obtaining an image of the whole substrate surface or a part of the substrate surface bearing fluorescent spots due to the fluorescent substances;
measuring an initial number of fluorescent spots and an initial intensity of each of the fluorescent spots of the image;
promoting to decrease the intensity of each of the fluorescent spots; and
estimating a number of molecules or a molecular density of the sample based on the initial number of fluorescent spots and the initial intensities of the fluorescent spots regarding a changing manner of the fluorescent intensity, the number of fluorescent spots or both in the process of promoting to decrease the intensity or after the intensity is decreased.

3. A method of measuring a number of molecules or a molecular density of a sample fixed on a substrate surface comprising steps of:
labeling each molecule of the sample with a preset number of fluorescent substance or substances before or after the sample is fixed on the substrate surface;
obtaining an image of the whole substrate surface or a part of the substrate surface bearing fluorescent spots due to the fluorescent substances;
measuring an initial intensity of the fluorescent spots of the image;
promoting to decrease the intensity of each of the fluorescent spots;
measuring a decreased number of fluorescent spots and a decreased intensity of the fluorescent spots; and
estimating a number of molecules or a molecular density of the sample based on the initial intensity of the fluorescent spots regarding a relationship between the number of fluorescent spots and the fluorescent intensity after the intensity is decreased.

4. The method according to claim 3, wherein the relationship between the number of fluorescent spots and the fluorescent intensity is represented by a calibration curve.

5. The method according to claim 4, wherein the calibration curve is linear.

6. The method according to claim 4, wherein the calibration curve is a curve of higher order function.

7. The method according to claim 2, wherein the decrease is promoted by irradiating a laser beam to the sample.

8. The method according to claim 3, wherein the decrease is promoted by irradiating a laser beam to the sample.

9. The method according to claim 2, wherein the decrease is promoted by using a chemical quenching method.

10. The method according to claim 3, wherein the decrease is promoted by using a chemical quenching method.
